(19) United States
(12) Patent Application Publication
(10) Pub. No.: US 2015/0208931 A1
(43) Pub. Date: Jul. 30, 2015

(54) METHOD AND DEVICE FOR QUANTIFYING HEART RATE VARIABILITY (HRV) COHERENCE

(71) Applicant: NITTO DENKO CORPORATION, Osaka (JP)

(72) Inventors: Kittipong Kasamsook, Singapore (SG); Juliana Chua, Singapore (SG); Kumar Senthil, Singapore (SG)

(73) Assignee: NITTO DENKO CORPORATION, Osaka (JP)

(21) Appl. No.: 14/423,351

(22) PCT Filed: Aug. 22, 2013
(86) PCT No.: PCT/SG13/00362

§ 371 Date: Feb. 23, 2015

Related U.S. Application Data
(60) Provisional application No. 61/692,450, filed on Aug. 23, 2012.

(51) Int. Cl. A61B 5/024; A61B 5/00; A61B 5/0402

(52) U.S. Cl. CPC A61B 5/02405; A61B 5/02416; A61B 5/0402; A61B 5/7246

(57) ABSTRACT

Method 100 and device for quantifying heart rate variability coherence of a subject is disclosed herein. The method 100 comprises obtaining a bio-signal (such as a PPG signal) from the subject at 102 and deriving a time-domain heart rate variability signal from the bio-signal at 108. Further, at 112, the method 100 further includes correlating the time-domain heart rate variability signal with a sine wave representing a time domain reference heart rate variability signal to obtain a correlated heart rate variability signal, and this includes adjusting frequency of the sine wave and performing cross-correlation between the sine wave at each of the adjusted frequencies and the heart rate variability signal to obtain the correlated heart rate variability signal. Further, at 116, the method includes quantifying the heart rate variability coherence based on the correlated heart rate variability signal.

This invention relates to a method and device for quantifying heart rate variability coherence, more particularly but not exclusively, for a human subject.

A healthy heart has a natural beat-to-beat variation in rate, known as Heart Rate Variability (HRV). Patterns and rhythms within this variability are important to health and well-being. Research shows that when you shift into a different emotional state, heart rhythms immediately change. Negative emotions such as anxiety and frustration show a disordered and chaotic variation. Positive emotions like tranquility shows an ordered rhythm synchronized with breathing.

The beat to beat variation is under the direct control of the sympathetic nervous system (SNS) and parasympathetic nervous system (PNS). The autonomic nervous system (ANS), comprises of both SNS and PNS, from which the system's response impacts our daily activities (e.g. your mood, your sense of touch).

The interaction of both SNS and PNS gives rise to Heart Rate Variability (HRV), which is a reflection of ANS balance and imbalance in the body.

Conventional HRV analysis requires 5 minutes of data collection which is in certain instances too time-consuming for users to undertake on a regular basis for understanding their well-being. Additionally, conventional use of HRV with Respiratory Rate (RR) for cross relation gives rise inaccuracy of results.

### SUMMARY

In a first aspect of the invention, there is provided a method of quantifying heart rate variability coherence of a subject, the method comprising
(i) obtaining a bio-signal from the subject;
(ii) deriving a time-domain heart rate variability signal from the bio-signal;
(iii) correlating the time-domain heart rate variability signal with a sine wave representing a time domain reference heart rate variability signal to obtain a correlated heart rate variability signal; and
(iv) quantifying the heart rate variability coherence based on the correlated heart rate variability signal;
wherein the correlating step (iii) includes
(iv) adjusting frequency of the sine wave;
(v) performing cross-correlation between the sine wave at each of the adjusted frequencies and the heart rate variability signal to obtain the correlated heart rate variability signal.

An advantage of the described embodiment is that it achieves a much quicker coherent result—estimated only 1 minute of data collection time. This is much quicker than conventional ways such as frequency domain HRV analysis which typically require 5 minutes to analyse the low frequency power spectral density (PSD) of the signals. Further, the use of frequency scanning method and cross-correlation, it is possible to identify strongest cross correlation between HRV and the ideal individual biofeedback signal (sine wave) to achieve HRV coherence.

Preferably, step (ii) may comprise obtaining an intermediate time-domain heart rate variability signal from the bio-signal; averaging the intermediate time-domain heart rate variability signal to obtain an average heart rate variability signal; and deriving the time-domain heart rate variability signal from the intermediate time-domain heart rate variability signal and the average heart rate variability signal. Preferably, the time-domain heart rate variability signal may be derived by subtracting the intermediate time-domain heart rate variability signal by the average heart rate variability signal.

In one example, averaging the intermediate time-domain heart rate variability may be performed over the HRV's entire time window. In an alternative example, the method may further comprise segmenting the HRV's time window into a plurality of intermediate time windows with each intermediate time window having a corresponding segmented HRV signal, and averaging the corresponding segmented HRV signals to obtain the average HRV signal.

The method may also further comprise deriving a strongest cross correlation from the correlated heart rate variability signal and a frequency corresponding to the strongest cross correlation. The method may further comprise calculating standard deviations of peak-to-peak of the bio-signal.

Preferably, quantifying the heart rate variability coherence may include calculating a wellness index based on the frequency corresponding to the strongest cross correlation, the percentage of the strongest cross-correlation and the standard deviation of the peak-to-peak of the bio-signal.

Advantageously, step (v) may include obtaining cross correlation coefficients r_{xy} based on the formula:

$${r}_{xy} = \frac{{\sum }_{i = 1}^{n} ( x ( i ) - \overline{x} ) ( y ( i ) - \overline{y} )}{\sqrt{{\sum }_{i = 1}^{n} {( x ( i ) - \overline{x} )}^{2} {\sum }_{i = 1}^{n} {( y ( i ) - \overline{y} )}^{2}}}$$

where,
x(i) is time series of a reference heart rate variability signal (sine wave);
x̅ is mean of the corresponding x(i) time series;
y(i) is time series of a heart rate variability signal obtained from a subject; and
y̅ is mean of the corresponding y(i) series; and
r_{xy} is the cross-correlation coefficient of x(i) and y(i) series.

The bio-signal from the subject may include a PPG signal or an ECG signal. The method may also include increasing or reducing the frequency of the sine wave by a predetermined interval. Specifically, the predetermined interval may be 0.005 Hz, or other suitable intervals.

In a second aspect of the invention, there is provided a device for quantifying heart rate variability coherence of a subject, the device comprising a processor configured to (i) obtain a bio-signal from the subject; (ii) derive a time-domain heart rate variability signal from the bio-signal; (iii) correlate the time-domain heart rate variability signal with a sine wave representing a time domain reference heart rate variability signal to obtain a correlated heart rate variability signal; and (iv) quantify the heart rate variability coherence based on the correlated heart rate variability signal; wherein the processor is further configured to (iv) adjust frequency of the sine wave; (v) perform cross-correlation between the sine wave at each of the adjusted frequencies and the heart rate variability signal to obtain the correlated heart rate variability signal.

It is envisaged that features related to one aspect may be relevant to the other aspect(s).

### BRIEF DESCRIPTION OF THE DRAWINGS

An exemplary embodiment of the invention will now be described with reference to the accompanying drawings in which:

FIG. 1 is a flow chart illustrating a method of qualifying heart rate variability coherence according to a preferred embodiment of the invention;

FIG. 2 is a pictorial representation of a cross correlation step using frequency scanning method used in the method of FIG. 1;

FIG. 3 is a graph illustrating results of the cross correlation of FIG. 2;

FIG. 4a illustrates a time window of a heart rate variability signal of the flow chart of FIG. 1;

FIGS. 4b and 4c illustrate the time window of FIG. 4a being segmented into a plurality of segmented time windows;

FIG. 5a illustrates a HRV signal for “Subject 2” which has a fluctuating baseline,

FIG. 5b illustrates a HRV signal for “Subject 5” which has a relatively constant baseline;

FIG. 6 is a graph illustrating results of adjusting the baseline of a HRV signal based on segmenting the time window of FIGS. 4b and 4c;

FIGS. 7a, 7b and 7c are reference tables for deriving a Zen index based on the cross correlation results of FIG. 3, and other parameters;

FIG. 8 shows how the Zen index may be used in combination with another index (Vita index for fitness) to represent overall well-being of the subject; and

FIG. 9 is a pictorial representation of how the cross-correlated graph of FIG. 3 may be used to train the subject to breathe in a particular manner to achieve particular coherent result.

### DETAILED DESCRIPTION

The two branches of the ANS (SNS and PNS) usually function in tandem with each other (i.e. when one is activated, the other is suppressed).

Division of SNS results in stress arousal (i.e. both positive and negative). During SNS arousal, increased heart rate and respiration, cold and pale skin, dilated pupils, raised blood pressure are expected symptoms.

Division of PNS results in states of rest and relaxation. During PNS arousal, decreased heart rate and respiration, warm and flushed skin, normally reactive pupils, lowered blood pressure are expected symptoms.

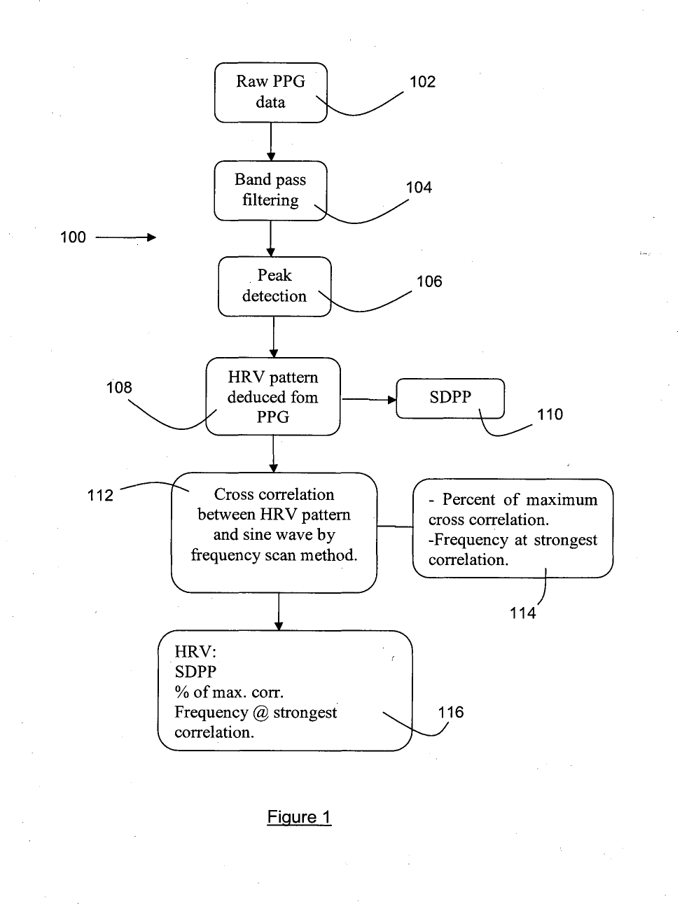

FIG. 1 is a flow chart illustrating a method 100 of qualifying HRV coherence, according to a preferred embodiment.

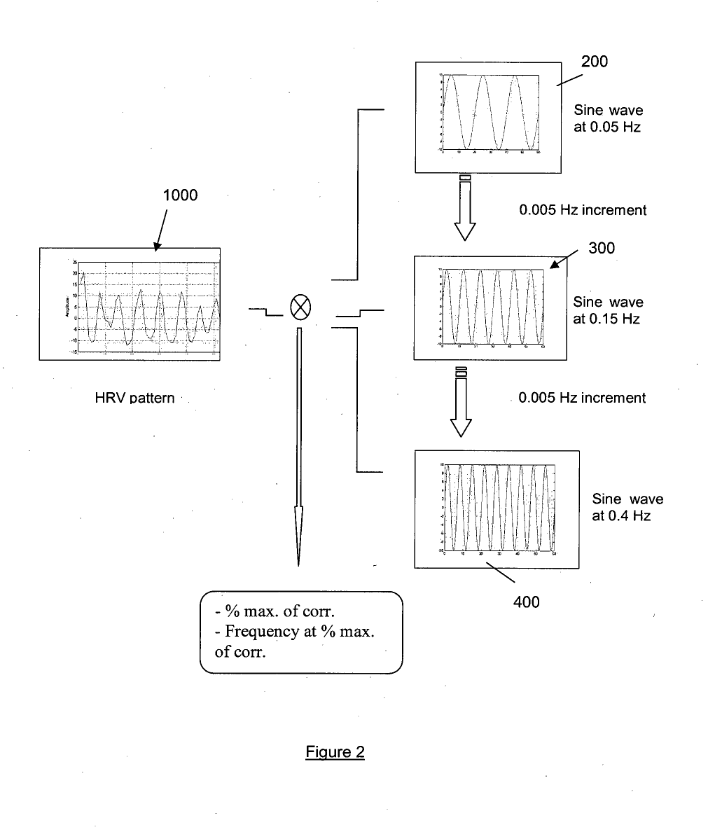

At 102, a bio-signal is obtained from a human subject, broadly referred to as a user of the method. In this embodiment, the user places his fingertip on a measurement device such as a combination of a mobile telephone and a measurement unit as disclosed in WO 2012/099534 (PCT/SG2011/000424), the contents of which are incorporated herein by reference, to obtain a PPG signal as the bio-signal. The measurement device includes a band pass filter to filter the obtained PPG signal at 104 to produce a filtered PPG signal. The measurement device also includes a peak detector and at 106, the peak detector detects peaks of the filtered PPG signal to produce a series of peak positions of the PPG signal and time indications corresponding to the series of peak positions. At 108, a processor of the measurement device derives heart rate variability (HRV) of the user from the series of peak positions and time indications at step 106 and this is illustrated as a HRV signal 1000 in FIG. 2. Further, standard deviation of the series of peak positions (i.e. peak-to-peak, SDPP) is also derived and this is shown in step 110.

At 108, average of the HRV signal is also obtained by averaging total data points of the HRV signal. Next, baseline of the HRV signal is adjusted to ground state by subtracting the HRV signal by the average HRV signal to produce a normalised HRV signal. This is to improve the accuracy of correlation which is the next step.

Next, at 112, cross correlation is performed between the normalised HRV signal and a sine wave 200 by frequency scanning method. This involves varying the frequency of the sine wave 200 from 0.05 Hz to 0.4 Hz with each increment of 0.005 Hz and at each interval, cross correlation with the normalised HRV signal is performed. To derive a series of cross-correlation coefficients as the cross-correlation result, mathematically, the formula for deriving the cross-correlation coefficients of the x and y series is as follows:

$${r}_{xy} = \frac{{\sum }_{i = 1}^{n} ( x ( i ) - \overline{x} ) ( y ( i ) - \overline{y} )}{\sqrt{{\sum }_{i = 1}^{n} {( x ( i ) - \overline{x} )}^{2} {\sum }_{i = 1}^{n} {( y ( i ) - \overline{y} )}^{2}}}$$

Where x̅ is mean of the corresponding x(i) time series;
x(i) is time series of a reference heart rate variability signal (sine wave);
y̅ is mean of the corresponding y(i) series;
y(i) is time series of a heart rate variability signal obtained from a subject; and
r_{xy} is the cross-correlation coefficient of x(i) and y(i) series.

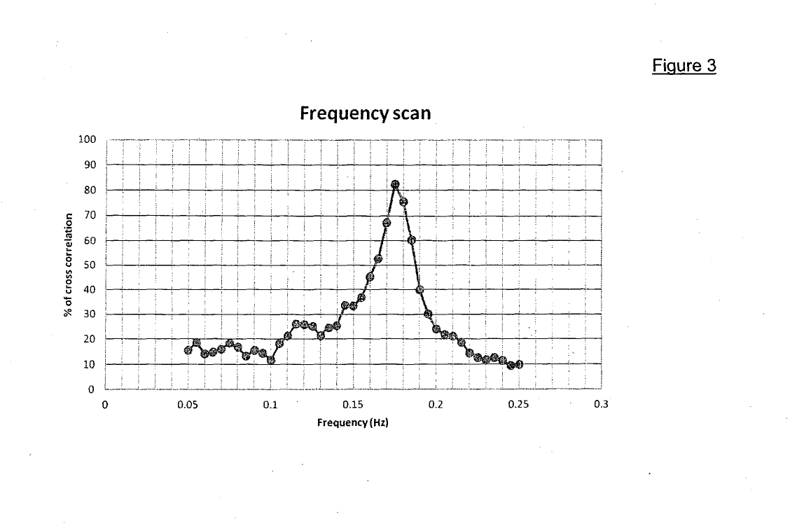

Referring to FIG. 2, graph 1000 illustrates the exemplary normalised HRV pattern/signal 1000 obtained from the user at step 108 above (reference FIG. 2), y(i). The graph 200 illustrates the reference HRV signal and in this example, it is a sine wave at 0.05 Hz, i.e. x(i). Next, frequency scanning method is used by increasing the sine wave at intervals of a predetermined frequency and in this case the increment is 0.005 Hz (see graph 300 of FIG. 2, which shows the sine wave being adjusted to 0.15 Hz and graph 400 with the sine wave at 0.4 Hz), and at each interval (or increment), cross-correlation is performed between the reference HRV signal and the HRV pattern 1000 of FIG. 2. The cross-correlated result is illustrated in FIG. 3.

It should also be appreciated that it is possible to obtain the strongest correlation based on the maximum % of cross correlation or peak of the graph in FIG. 3 and the corresponding frequency, and this is performed at step 114. In the example of FIG. 3, the peak is obtained at a frequency of 0.175 Hz. In other words, if this subject is able to control his/her breathing (or respiratory rate) to achieve this frequency of 0.175 Hz, the subject would achieve maximum coherence which may be regarded as the best coherence, i.e., ability to follow sine wave closely, for this subject.

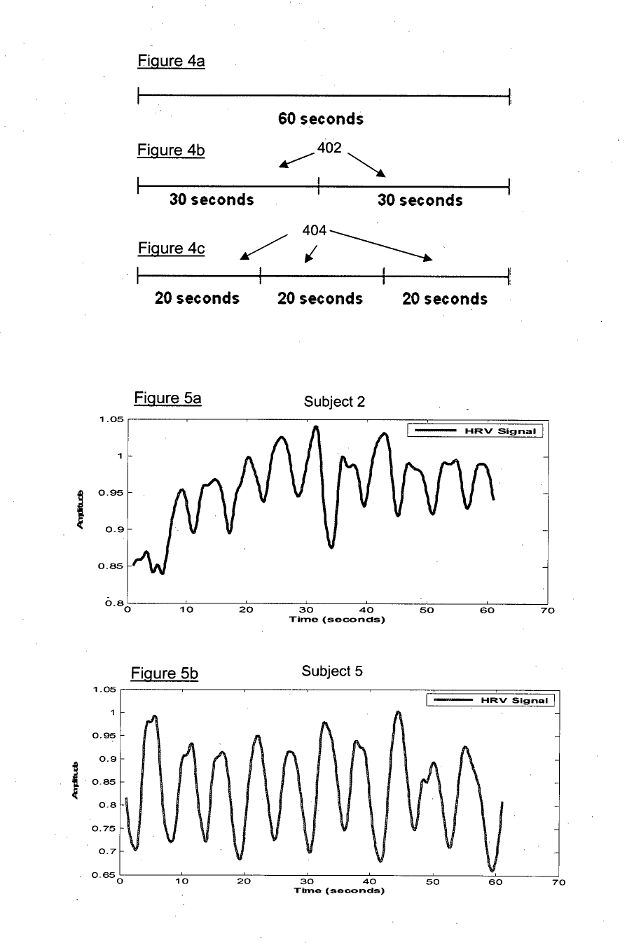

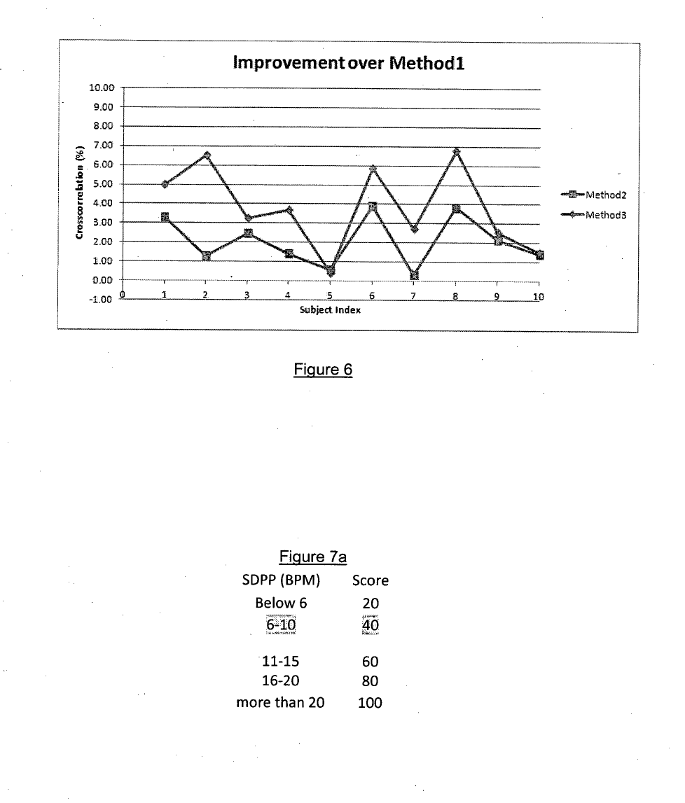

As explained above, at 108, the average of the HRV signal is obtained by averaging total data points of the HRV signal and thereafter, the baseline of the HRV signal is adjusted to ground state by subtracting the HRV signal by the average HRV signal to produce the normalised HRV signal. In other words, averaging of the HRV signal is performed throughout an entire time window of the HRV signal for example, 60 seconds as that shown in FIG. 4a.

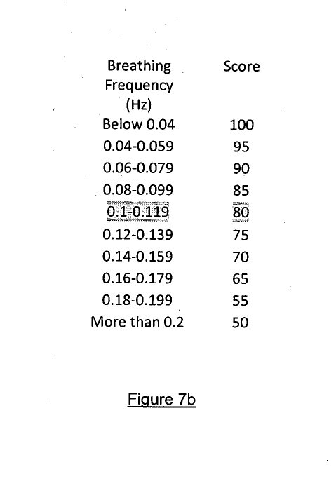

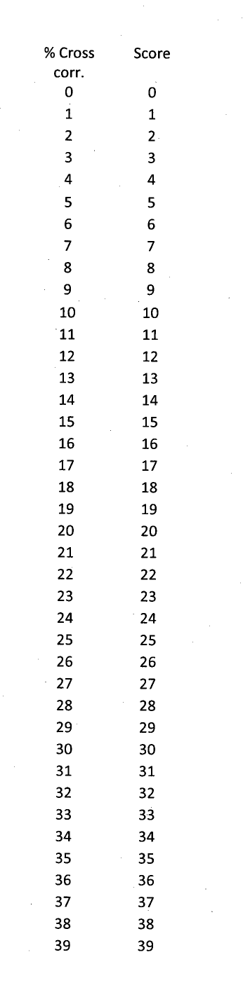

It is found that different people, however, may have different HRV signals, in particular different baselines. For example, FIG. 5a illustrates a HRV signal for “Subject 2” which has a fluctuating baseline, whereas FIG. 5b illustrates a HRV signal for “Subject 5” which has a relatively constant baseline. In other words, the baseline of HRV patterns for individual subjects may vary depending on their conditions during the measurement period, it is observed that processing of an entire signal (FIG. 4a) as is may degrade the accuracy of the cross correlation.

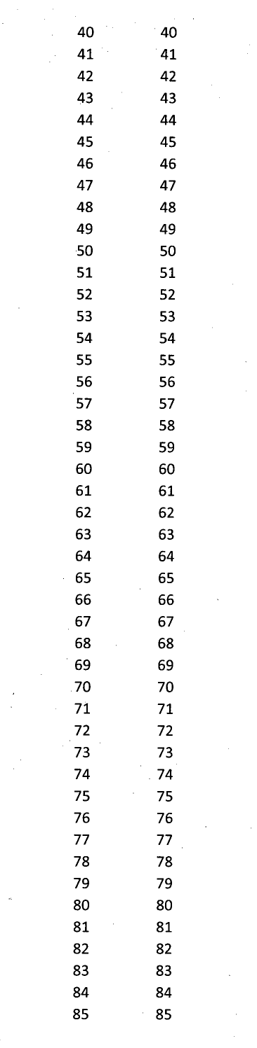

In order to improve the cross correlation's accuracy at step 112, an additional step is preferably performed to frame or segment the HRV signal's time window into a plurality of window such as two or more windows. As an example, FIG. 4b and FIG. 4c show the HRV signal of FIG. 4a having a time window of 60 seconds being framed into multiples of 30-second segmented windows 402 as well as multiples of 20-second segmented windows 404 respectively with each segmented time window 402,404 having a corresponding segmented HRV signal. Averaging of the HRV signal is then performed within each segmented time window 402,404 and the baseline of the HRV signal adjusted accordingly. The result is shown in FIG. 6 for different subjects (i.e. users) namely, Subjects 1 to 10 (see x-axis of FIG. 6) which demonstrates significant improvements when framing is used. Specifically, Method 1 mentioned in FIG. 6 refers to the averaging of the HRV signal without framing i.e. FIG. 4a whereas Methods 2 and 3 correspond to the framing methods proposed in FIGS. 4b and 4c respectively. To further elaborate, y-axis of FIG. 6 represents percentage difference between. Methods 2/3 and Method 1 and using Subject 2 (i.e. FIG. 5a) as an example (see “2” in the x-axis), Subject 2 shows an improvement of ˜1% when using Method 2 and 6.5% when using Method 3 over Method 1. Therefore, if the percentage of cross-correlation is 60% after applying Method 1, the percentage of correlation will be 61% for Method 2 and 66.5% for Method 3, thus increasing the accuracy of the data collated.

As it can be appreciated, with the proposed HRV analysis method, which is performed in time domain, it is possible to quantify the coherence between HRV and breathing pattern more quickly, for example in around 1 minute as opposed to conventional HRV analysis method using both frequency and time domain which typically takes 5 mins. It is also possible to achieve a more accurate coherence by identifying percentage of maximum cross correlation and frequency at strongest correlation.

The cross-correlation results between Heart Rate Variability (HRV) & individual biofeedback signal of subject, along with the Standard Deviation of Peak-to-Peak PPG (SDPP, Y-axis), are used to interpret the Autonomic Nervous System's (ANS) activities at step 116 of FIG. 1, and HRV coherence is based on three preferred features:
(1) Standard deviation of peak-to-peak (SDPP) of the PPG signal (obtained from step 110);
(2) Frequency at the strongest (max.) cross correlation;
(3) Percentage of strongest cross-correlation.

With the use of the filtered PPG signal from the individual subject and biofeedback signal; together with the SDPP, percentage of strongest cross-correlation and frequency of strongest correlation between HRV and biofeedback signal, it is possible to quantify the HRV coherence more precisely by applying the following algorithm where the values of weighting factors a, b, c can be predetermined and in this embodiment, the weighting factors, a, b and c are “0.25”, “0.25” and “0.5” respectively.

Zen Index=a(SDPP)+b(Frequency)+c(Percentage of strongest cross-correlation)

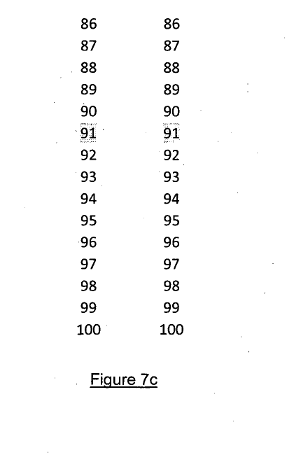

As an example, a subject with the following measured results will be processed by weighting factors derived from reference tables shown in FIGS. 7a, 7b and 7c prior to arriving at the final Zen Index score of:

Zen Index=10+20+45.5=75.5
Variation of heart rate (SDPP) in the range of 6-10 BPM=40 (from FIG. 7a)
Breathing frequency in the range of 0.1-0.119 Hz=80 (from FIG. 7b)
Percentage of cross correlation coefficient between HRV pattern and simulated bio-feedback signal of 91=91 (from FIG. 7c)

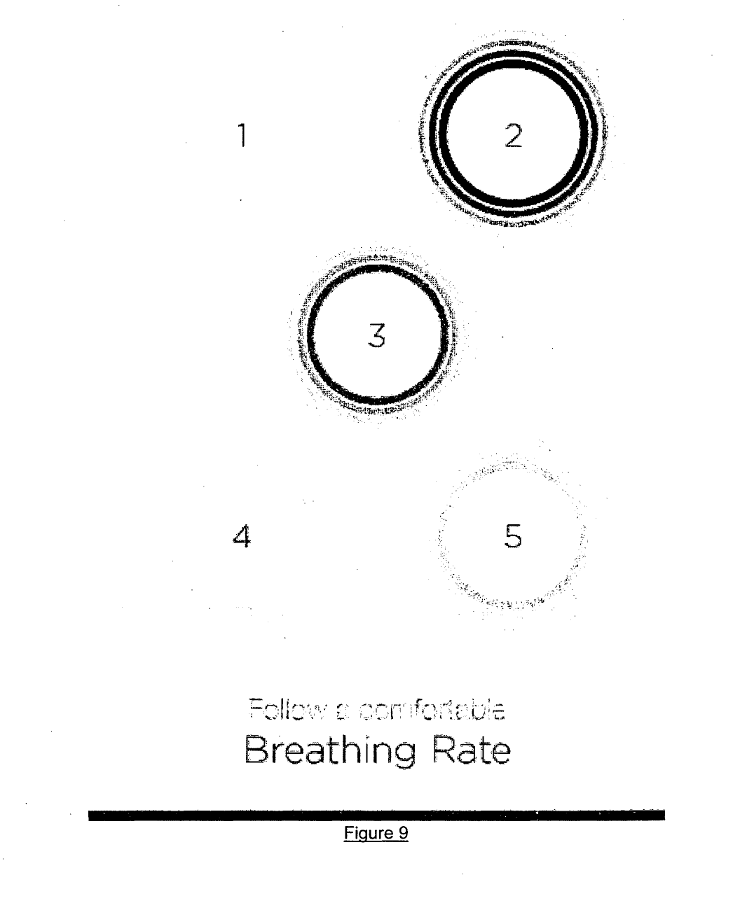

To elaborate, values of the “score” in the tables in FIGS. 7a to 7c are derived by defining the minimum and maximum range for each parameter, and dividing the total range over 100 points. For example, for frequency, the lower the frequency, the better the score. Therefore, any value below 0.04 Hz is given a score of 100 because it is the lowest frequency in the LF band. Based on the breathing training guide (FIG. 9), the current embodiment uses five cycles, 0.0583 Hz, 0.0833 Hz, 0.117 Hz, 0.167 Hz and 0.217 Hz. Therefore, any values more than 0.2 Hz will be given a score of 50.

As for SDPP, it is appreciated that the higher BPM (Higher Heart Rate Variation), the better the score, and the minimum HRV should be around 5 BPM for normal people. Therefore the lowest score is set as values less than 6 BPM. For maximum SDPP, the maximum peak of heart rate swing was calculated on 25% of 40 BPM averaged heart rate. Thus, the peak-to-peak swing should be 20 BPM. Therefore any values more than 20 will get 100 score.

Derivation of the score of percentage of strongest cross correlation should be self-explanatory from FIG. 7c.

Based on the above examples, it should be appreciated that the Zen Index is thus=a (SDPP)+b (Frequency)+c (% of cross correlation)=(0.25×40)+(0.25×80)+(0.5×91)=10+20+45.5=75.5.

As it can be appreciated from the above, the results from the cross-correlated graph of FIG. 3 may be used to derive the “Zen” index which may be an indication or representation of the mood or well-being status of the subject, in particular based on the maximum % of cross correlation.

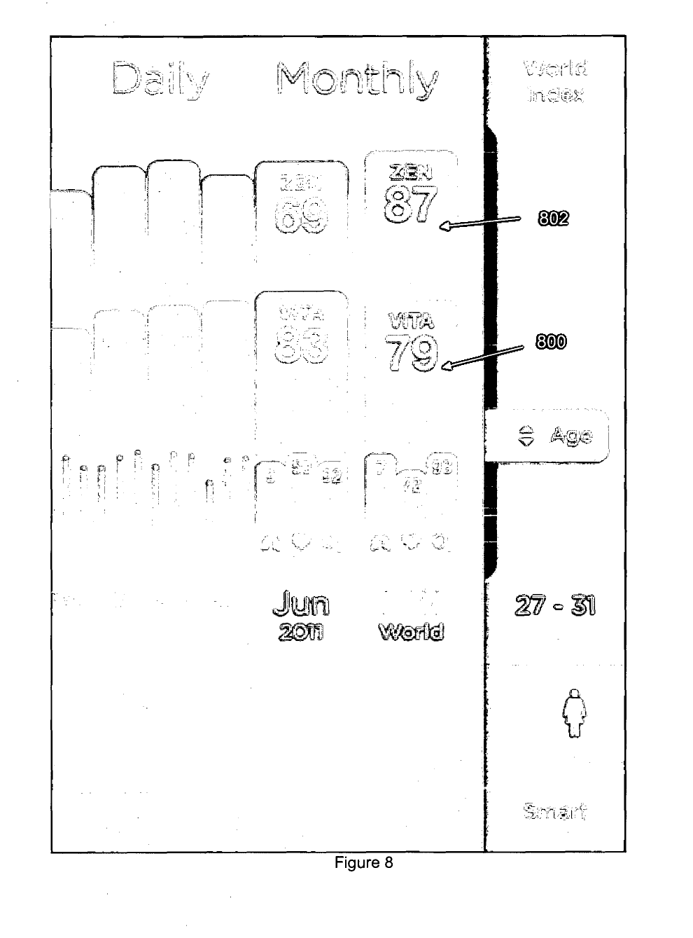

Further, this Zen index may be used together with other index to determine well-being status of the subject. For example, the Zen index may be used in combination with the disclosure of WO 2012/099534 (PCT/SG2011/000424), the content of which is incorporated herein by reference, to provide a well-being status of the subject. FIG. 8 illustrates of the combined use of a fitness index 800 and the Zen index 802 for a subject, where the comparison was made to the total users/subjects herein defined as world index and further breakdown into age groups.

Further, the cross-correlated graph of FIG. 3 may be used to train the subject to breathe in a particular manner to achieve a particular coherent result or perhaps, which controlled breathing pattern is the optimum to give a best coherence. It is possible that the cross-correlated graph may be used as a well-being guide for subjects. For example, multiple frequencies that can be represented in various shapes onscreen, with an aim to resemble or represent that of sine waves so as to achieve guiding a subject through a controlled breathing pattern. The multiple frequencies in this case could be represented for instance by concentric circles marked by “1”, “2”, “3”, “4” and “5”, as shown in FIG. 9, where each circle expands and collapses in accordance to a range of breathing frequency and in this embodiment, the numbers correspond respectively to 0.0583 Hz, 0.0833 Hz, 0.117 Hz, 0.167 Hz and 0.217 Hz.

Similarly in the same fashion, the visual guide of concentric circles may be replaced by an audio guide, with an aim to resemble or represent that of sine waves so as to achieve guiding a subject through a controlled breathing pattern. The multiple frequencies in this case could be represented for instance by soothing music together with an instructional guide, where each instructional command to breath in and out are in accordance to a range of breathing frequency and in this embodiment, the numbers correspond respectively to 0.0583 Hz, 0.0833 Hz, 0.117 Hz, 0.167 Hz and 0.217 Hz.

To assess actual emotional state of our autonomic nervous system, the subject selects one the many frequencies presented on-screen, one that best suits current breathing cycle and breaths accordingly. For illustrative purpose, the subject when performing this measurement of controlled breathing is guided by one of the concentric circles or one of the audio guides that he/she selects. The benefits of the aided breathing, in this example via concentric circle or instructional command, enable the subject to be in better coherence so as to achieve an improve heart rate variability.

The same interface of FIG. 9 (or the audio guide) may also enable the subject to conduct breathing exercise with the benefit of improving overall well-being. For example with respect to the visual guide, instead of selecting the concentric circle that is closest to his current breathing, the subject selects a concentric circle that promotes deep/long breathing. In another example with respect to an audio guide, the subject similarly can select a routine that promotes deep/long breathing. The latter breathing exercise when performed on a regular basis regulates and improve breathing patterns to provide an enhance amount of oxygen to the body.

The described embodiment should not be construed as limitative. Instead of the PPG signal, and ECG signal may also be used or other plethysmograph signals may also be used.

Further, instead of starting from 0.05 Hz and increase the frequency of the sine wave (or broadly a reference HRV signal/pattern) by predetermined increments to 0.4 Hz, it is envisaged that the cross-correlation may begin at the upper limit of 0.4 Hz and the frequency of the sine wave is reduced by predetermined amounts to the lower end of 0.05 Hz. In other words, the frequency of the sine wave may be adjusted accordingly. Likewise, the upper limit of 0.4 Hz and the lower limit of 0.05 Hz may be varied, and amount of adjustment may be varied too, and not fixed at 0.005 Hz (although this is preferred).

1. A method of quantifying heart rate variability coherence of a subject, the method comprising;
(i) obtaining a bio-signal from the subject;
(ii) deriving a normalised time-domain heart rate variability signal from the bio-signal;
(iii) correlating the normalised time-domain heart rate variability signal with a sine wave representing a time domain reference heart rate variability signal to obtain a correlated heart rate variability signal; and
(iv) quantifying the heart rate variability coherence based on the correlated heart rate variability signal;
wherein the correlating step (iii) includes:
(v) adjusting frequency of the sine wave; and
(vi) performing cross-correlation between the sine wave at each of the adjusted frequencies and the heart rate variability signal to obtain the correlated heart rate variability signal.

2. A method according to claim 12, wherein averaging the intermediate time-domain heart rate variability is performed over the HRV's entire time window.

3. A method according to claim 12, further comprising;
segmenting the HRV's time window into a plurality of intermediate time windows with each intermediate time window having a corresponding segmented HRV signal, and
averaging the corresponding segmented HRV signals to obtain the average HRV signal.

4. A method according to claim 1, further comprising deriving a strongest cross correlation from the correlated heart rate variability signal and a frequency corresponding to the strongest cross correlation.

5. A method according to claim 4, further comprising calculating standard deviations of peak-to-peak of the bio-signal.

6. A method according to claim 5, wherein quantifying the heart rate variability coherence includes calculating a wellness index based on the frequency corresponding to the strongest cross correlation, the percentage of the strongest cross-correlation and the standard deviations of the peak-to-peak of the bio-signal.

7. A method according to claim 1, wherein step (v) includes obtaining cross correlation coefficients r_{xy} based on the formula:
$${r}_{xy} = \frac{{\sum }_{i = 1}^{n} ( x ( i ) - \overline{x} ) ( y ( i ) - \overline{y} )}{\sqrt{{\sum }_{i = 1}^{n} {( x ( i ) - \overline{x} )}^{2} {\sum }_{i = 1}^{n} {( y ( i ) - \overline{y} )}^{2}}}$$
where,
x(i) is time series of a reference heart rate variability signal (sine wave);
x̅ is mean of the corresponding x(i) time series;
y(i) is time series of a heart rate variability signal obtained from a subject; and
y̅ is mean of the corresponding y(i) series; and
r_{xy} is the cross-correlation coefficient of x(i) and y(i) series.

8. A method according to claim 1, wherein the bio-signal from the subject includes a PPG signal or an ECG signal.

9. A method according to claim 1, wherein step (iv) includes increasing or reducing the frequency of the sine wave by a predetermined interval.

10. A method according to claim 9, wherein the predetermined interval is 0.005 Hz.

11. A device for quantifying heart rate variability coherence of a subject, the device comprising a processor configured to:
(i) obtain a bio-signal from the subject;
(ii) derive a normalised time-domain heart rate variability signal from the bio-signal;
(iii) correlate the normalised time-domain heart rate variability signal with a sine wave representing a time domain reference heart rate variability signal to obtain a correlated heart rate variability signal; and
(iv) quantify the heart rate variability coherence based on the correlated heart rate variability signal;
wherein the processor is further configured to:
(v) adjust frequency of the sine wave; and
(vi) perform cross-correlation between the sine wave at each of the adjusted frequencies and the heart rate variability signal to obtain the correlated heart rate variability signal.

12. A method according to claim 1, wherein step (ii) comprises:
obtaining an intermediate time-domain heart rate variability signal from the bio-signal;
averaging the intermediate time-domain heart rate variability signal to obtain an average heart rate variability signal; and
subtracting the intermediate time-domain heart rate variability signal by the average heart rate variability signal to adjust baseline of the intermediate time-domain heart rate variability signal in order to obtain the normalised time-domain heart rate variability signal.

13. A device according to claim 11, wherein to derive the normalised time domain heart rate variability signal at step (ii), the processor is further configured to:
obtain an intermediate time-domain heart rate variability signal from the bio-signal;
average the intermediate time-domain heart rate variability signal to obtain an average heart rate variability signal; and
subtract the intermediate time-domain heart rate variability signal by the average heart rate variability signal to adjust baseline of the intermediate time-domain heart rate variability signal in order to obtain the normalised time-domain heart rate variability signal.
